(19) United States
(12) Patent Application Publication
(10) Pub. No.: US 2025/0391529 A1
(43) Pub. Date: Dec. 25, 2025

(54) SYSTEM AND METHOD FOR IMPROVING THE REVIEW AND REPORTING OF BIOELECTRICAL DATA

(71) Applicant: Healthcare Innovation Technologies, Inc., Lewes, DE (US)

(72) Inventor: Thomas W. Komorowski, Brick, NJ (US)

(21) Appl. No.: 18/751,316

(22) Filed: Jun. 23, 2024

(51) Int. Cl. G16H 15/00; G16H 50/70

(52) U.S. Cl. CPC G16H 15/00; G16H 50/70

(57) ABSTRACT

A system and method for improving the quality and speed of physician review of data, the system and method having a first user portal for sharing data and receiving reports and a second user portal for data input, automatically generating custom reports and medical insights. The custom reports are automatically generated through the assignment of one or more preset non-opaque classification buttons to the data.

### BACKGROUND

### Field on Invention

The present invention relates to systems and methods for improving the quality and speed of review and reporting of bioelectrical data.

### Related Art

Currently in the medical field, increasing global economic pressure has resulted in the need for physicians to increase the volume of patients seen in order to maintain economically sustainable practices. However, during a patient visit a physician generally needs to take time to gather data from the patient, provide a diagnosis, and discuss possible treatment options with the patient. By increasing the volume of patients seen physicians are forced to spend less time with each patient which is not feasible in every situation and may lead to ineffective services.

Attempts to resolve this problem have been made through the creation of software systems. In the art, current software systems conduct computer analysis of patient data providing the physician with a proposed diagnosis for the physician to either simply approve or reject. This reduces the time the physician spends reviewing each patient’s data thereby allowing the physician to increase patient volume. It is common knowledge that humans tend to have an inherent bias towards computer-generated results. What is needed is a system and method of improved review of bioelectrical data and other health data which reduces the time it takes to provide diagnosis and treatment options, allows the physician to conduct more patient visits per day, eliminates the need for a physical office, billing department, front desk staff and emergency service scheduling, and increases the quality of the information shared between patient and physician.

The present invention discloses a system and method for improving interaction, review, and reporting of bioelectrical data through the use of an interactive single screen view on a user interface of a device, the system and method configured to automatically generate customized medical diagnosis reports. By leveraging multiple vectors of bioelectrical data and enhancing physician interactions with this data, the invention creates a platform that supports more accurate and faster diagnosis of symptoms. Additionally, it streamlines physicians' workflows and improves communication with patients, fundamentally transforming how patients interact with the healthcare system.

### SUMMARY OF INVENTION

It is to be understood that the phraseology and terminology employed herein are for the purpose of the description and should not be regarded as limiting.

The present invention is directed to an application and method for facilitating the review, analysis, and reporting of bioelectrical data. The application includes a first user portal, the first user portal having a means for importing bioelectrical data and vital signs and/or symptoms and communication from one or more third-party applications or devices, a means for sharing the bioelectrical data with a second user, a means for inputting symptom information, and a means for receiving a diagnosis report, wellness guidance, and primary and/or secondary prevention guidance. The application also includes a second user portal, the second user portal having a means for receiving bioelectrical data from a first user, a means for receiving symptom information from a first user, a means for automatically creating a custom personalized diagnosis report, and a means for sharing the diagnosis report. The means for automatically creating a custom diagnosis report comprises diagnosis buttons for assignment to the bioelectrical data charts.

These and other features of the present invention will become readily apparent upon further review of the specification and drawings.

### BRIEF DESCRIPTION OF THE DRAWINGS

Embodiments of the present invention will be described by way of example only, and not limitation, with reference to the accompanying drawings. The drawings are not necessarily drawn to scale and wherever possible, the same or like reference numbers are used throughout the drawings to refer to the same or like features.

FIG. 1 illustrates an example user screen showing accessibility to a first user portal and a second user portal in accordance with an embodiment of the present invention.

FIG. 2 illustrates an example user screen of a first user portal of the present invention.

FIG. 3A illustrates an example user screen showing the single review screen of the second user portal.

FIG. 3B illustrates an example user screen showing the single review screen of the second user portal where a bioelectrical data chart is in a zoomed-in position.

FIG. 4 illustrates an example first user screen showing a means for inputting symptom information via the first user portal.

FIG. 5 illustrates an example user screen showing the single review screen of the second user portal where a bioelectrical data chart is in a zoomed-in position and categories and subcategories are assigned to the bioelectrical data chart.

FIG. 6 illustrates an example user screen showing the single review screen of the second user portal where a bioelectrical data chart is in a zoomed-in position and categories and subcategories are in the process of being assigned to the bioelectrical data chart.

FIGS. 7A - 7C illustrate example user screens showing a customized automatically generated medical diagnosis report.

FIG. 8 illustrates an example user screen showing notifications received by a patient user.

FIG. 9 illustrates an example user screen showing a records page of the first user portal.

### DETAILED DESCRIPTION

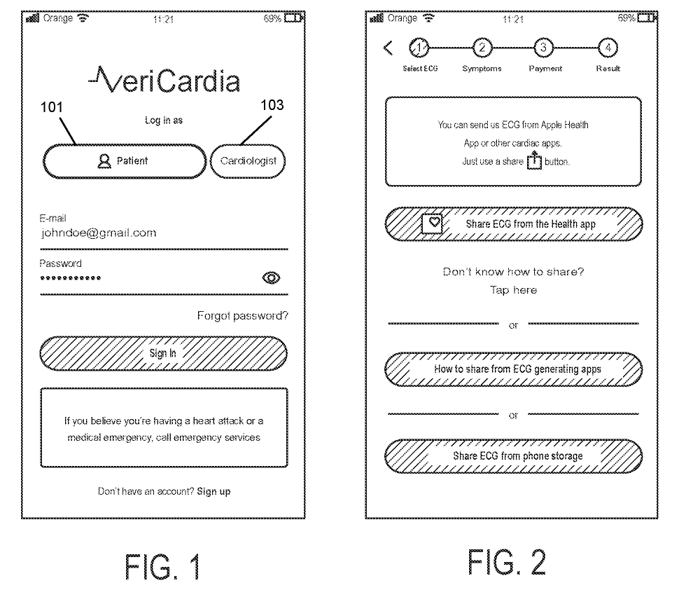

Referring now to the figures where similar reference characters denote similar elements throughout the figures, FIGS. 1 through 9 show preferred embodiments of the present invention. Specifically, FIGS. 1 through 9 illustrate a system and method for facilitating analysis of bioelectric data provided by a first user. Said bioelectrical data may include Electrocardiogram (ECG), Electroencephalogram (EEG), Galvanic skin response (GSR), Electromyogram (EMG), Electrooculography (EOG), and Mechanomyogram (MMG) data, as well as any other types of bioelectrical data known in the art. The system comprising of an application preferably in the form of computer software available for use, via download or otherwise on a mobile device. The application may also be in the form of a desktop application or a non-downloadable software as a service application. The application having a first user portal 101 and a second user portal 103. The first user portal 101 providing a first user with access to the application and the second user portal 103 providing a reviewing user with access to the application. A first user may be a patient or other individual providing health data through the first user portal 101 and the reviewing user may be a physician, medical technician, or other user reviewing the first users’ health data through the second user portal 103. The first user portal 101 and second user portal 103 may require user authentication in order to access the application. User authentication may be in the form of a username and a password, biometric input, or other form of user authentication known in the art. The functionalities of the application that are accessible to a first user through the first user portal 101 differ in part from the functionalities of the application that are accessible to a reviewing user through the second user portal 103. By way of example only, through the second user portal 103 a reviewing user may be able to manipulate bioelectrical data reports by adding notes or diagnosis buttons to the reports, or other forms of manipulation. Through the first user portal 101 a first user may not be able to manipulate their bioelectrical data reports in the same manner. Other examples of functionalities that may be available in the second user portal but not the first user portal include the ability to access and review data uploaded by multiple first users, the ability to access graphs and charts showing medical history and data trends, and the ability to view mean values (e.g. blood pressure, heart rate, etc.).

Upon accessing the system and method through the first user portal 101, a first user may import bioelectrical data and vital signs and/or symptoms via a Health Insurance Portability and Accountability Act compatible channel into the application from third-party applications or devices. For example, many mobile devices and smart watches include a ‘health application’ or similar applications for tracking a user’s health data. Bioelectrical data and vital signs and/or symptoms recorded on these third-party applications or devices may be imported into the application using a ‘share to’ feature on the mobile device or by any other means commonly known in the art for sharing data and files between devices and applications. FIG. 2 shows an exemplary user screen for importing bioelectrical data into the application. Vital signs and symptoms may include, without limitation, blood pressure, heart rate, heart rate variability, weight and body composition, body mass index, or self-reported symptoms. The types of third-party devices that this data may be obtained from include, without limitation, smart watches and bands, smart scales, blood pressure devices, and other devices known in the art.

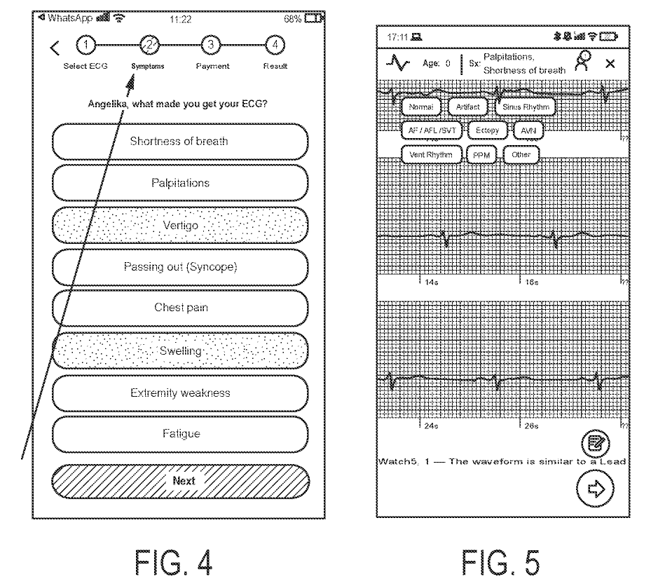

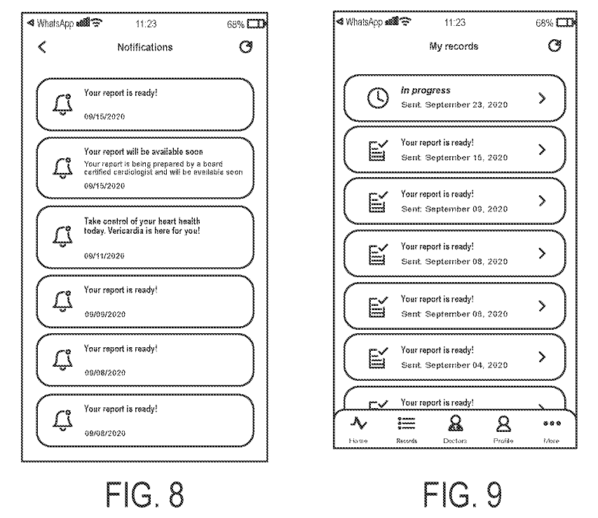

In embodiments of the present invention, the reviewing user may be prompted to input symptoms manually, through selection from a data inputs list as shown by way of example in FIG. 4, or through a combination thereof. The bioelectrical data and symptoms are shared by the first user or by the system for review by a reviewing user. After submission, the first user may be notified of the status of the review through push notifications to the first user’s mobile device as shown by way of example in FIG. 8. The first user may also opt to receive desktop notifications where the diagnosis application is used through a web or software as a service application, or by email, or text message. Through the first user portal 101 there may be a records page where a first user may access their older reports and records as shown by way of example in FIG. 9.

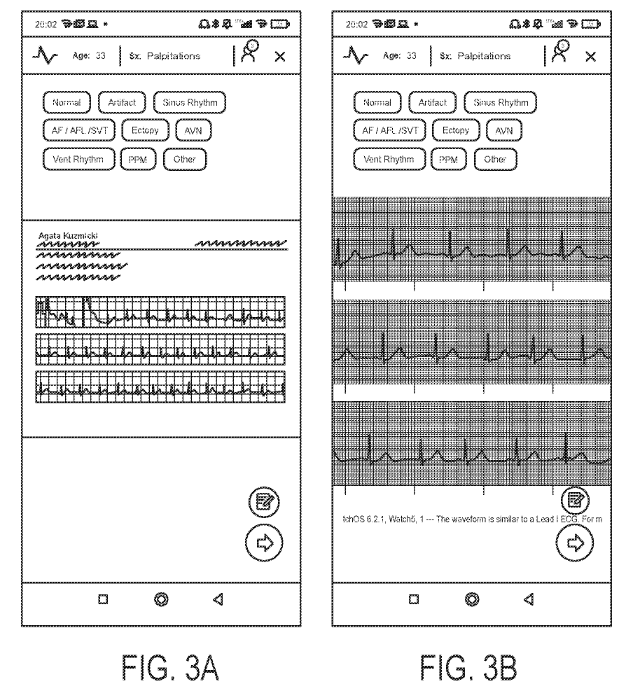

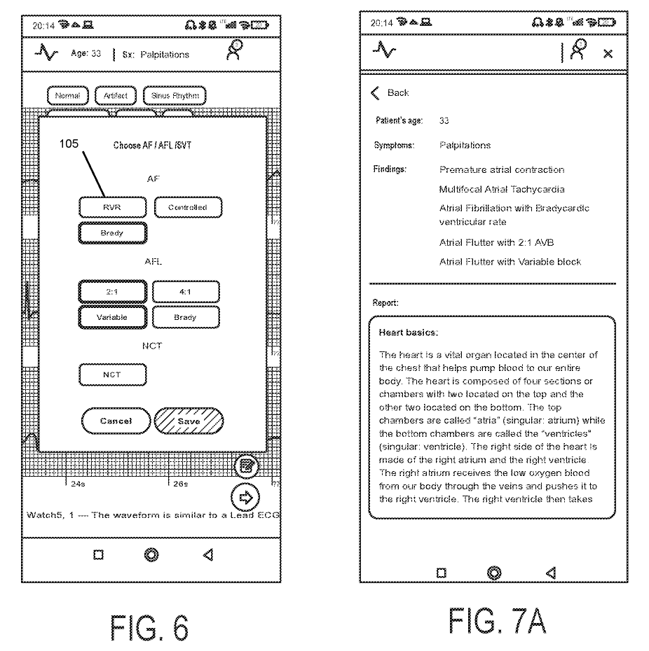

A reviewing user receives a notification through the application that there is bioelectrical data and vital signs and/or symptoms awaiting their review. Upon accessing the system and method through the second user portal 103 the reviewing user is able to view the first user’s bioelectrical data and vital signs and/or symptoms on a single review screen as shown by way of example in FIGS. 3A and 3B. The reviewing user reviews the bioelectrical data and vital signs and/or symptoms and using their professional judgment, selects one or more buttons from a plurality of pre-set classification buttons accessible through that single review screen of the application as shown by way of example in FIG. 6. In a preferred embodiment, the classification buttons are non-opaque. The non-opaque classification buttons are sufficiently translucent such that a physician user may view the contents of the single review screen beneath the non-opaque classification buttons. However, the non-opaque classification buttons may also be transparent. In a preferred embodiment of the present invention, the buttons comprise of text on a non-opaque background. This further facilitates the single screen review disclosed by the present invention which effectively shortens physician review time. The reviewing user assigns one or more of the one or more pre-set non-opaque classification buttons to appropriate locations on the bioelectrical data chart as shown by way of example in FIG. 5. This assignment may be done by dragging and dropping the buttons to appropriate locations on the bioelectrical data chart.

Pre-set non-opaque classification buttons may be arranged in categories 105 and some categories may also include subcategories. For example, where the bioelectrical data is in the form of an ECG chart, one category may be Atrial Fibrillation and its subcategories may be Atrial Fibrillation with Rapid Ventricular Rate, Atrial Fibrillation with Controlled Ventricular Rate, and Atrial Fibrillation with Bradycardic Ventricular Rate. Each pre-set non-opaque classification button is associated with report content. Report content may comprise of a textual description of the diagnosis and in some instances may also be accompanied by audio or audiovisual content. Pre-set non-opaque classification buttons and their associated content are stored in one or more databases.

The reviewing user may also select one or more non-opaque task buttons from pre-set non-opaque task buttons on the single review screen. The non-opaque task buttons are sufficiently translucent such that a reviewing user may view the contents of the single review screen beneath the non-opaque task buttons. However, the non-opaque task buttons may also be transparent. Each of the one or more non-opaque task buttons initiates a series of tasks when selected. For example, based on the diagnosis made by the reviewing user, the reviewing user may recommend that the first user schedule an in-person or telemedicine appointment and may also prescribe certain medications prior to the appointment. By selecting a single button the first user, when in receipt of the diagnosis report, is also prompted to schedule an appointment through the application and a prescription is sent to the first user’s pharmacy as recorded in the application. If the first user does not have a preferred pharmacy identified in the application, they are prompted to enter one at that time.

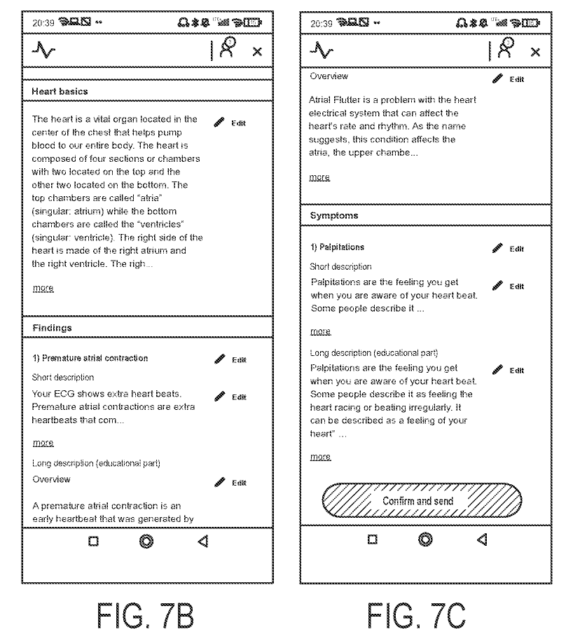

Using the same ECG example above, once all buttons are applied to the ECG chart, the reviewing user has the option to review the report that is automatically generated based on the report content associated with the pre-set non-opaque classification buttons assigned to the chart. FIGS. 7A, 7B, and 7C show exemplary screens of the automatically generated diagnosis report.

The system orders the report content based on relevancy scores generated by algorithms. These scores are assigned based on the urgency and appropriateness of various medical actions based on current patient data and historical interactions. In a preferred embodiment, the actions with the top three highest scores may be presented in the report as suggestions for the next steps. However, more than the top three may be presented. The reviewing user may edit the report. Once the report is satisfactory to the reviewing user, they may send the report to the first user who is notified through push notifications to the first user’s device on which the application is accessible. The first user may also opt to receive desktop notifications where the application is used through a web or software as a service application, or by email, or text message. In an embodiment of the invention, the system may use the relevancy scores to produce a list of tasks and/or multiple patients’ data for the reviewing user to review in order of priority. Either the first user or the reviewing user may send medical data from the system to a non-user such as a third party medical professional or facility.

In embodiments of the invention, the system and method allows both the first user and the reviewing user to access and/or receive automatically generated insights. These insights may be accessed within the first user portal and second user portal and/or may be sent to the user by email, text message, or other electronic means. Within the portal these insights may be accessed through certain interactions with the first user's medical data. By way of example only, this may be by hovering over certain data or clicking certain data. These automatically generated insights may be in the form of text, audio, or audio visual content and may include suggestions on lifestyle changes, medication changes, research on how certain lifestyle changes may affect lifespan, other medical research, and educational content.

In a preferred embodiment, a hybrid database architecture is used to optimize both data storage and performance. Persistent data such as patient demographics, medical records, and treatment histories are stored in a relational database management system (RDBMS) to ensure secure and ACID-compliant transactions. For dynamic user interactions and real-time analytics, a NoSQL database is used to enhance system responsiveness. Complex relationships within patient medical histories are managed using a graph database, facilitating high-performance analysis. Report content-to-button mapping is driven by generative AI models that analyze tokenized patient data. These generative AI models recommend medical actions and diagnostics through dynamic linking to interface buttons developed in backend algorithms. The AI models are trained on cardiovascular-specific datasets. This training involves analyzing provider actions,patient information, biodata, and self-reported data to enhance the accuracy of next-step recommendations and suggested test, and AI-medical decision system. The system may suggest additional tests either during or before a medical appointment, aiding in comprehensive and proactive patient care. The specificity of the training data provides improved support in clinical decision-making, where the data reviewer provides constant refinement of the outcome of data analysis, thus creating a unique multiuser refinement model.

What is claimed is:

1. An application for improving the quality and speed of review and interaction with data, comprising:
a) a first user portal, the first user portal configured to:
a. receive data from one or more third-party applications or devices;
b. receive data through manual user input;
c. share data with a second user;
d. receive a personalized report from the second user; and
e. receive automatically generated insights; and
b) a second user portal, the second user portal configured to:
a. receive data from a first user;
b. view said data on a single review screen wherein said single review screen comprises one or more classification buttons;
c. associate one or more of the one or more classification buttons with the data wherein said association is configured to automatically generate a report;
d. share the report with the first user; and
e. receive automatically generated insights.

2. The application of claim 1, wherein the classification buttons are non-opaque.

3. The application of claim 1, wherein the data comprises bioelectrical data.

4. The application of claim 1, wherein the data comprises bioelectrical data and symptom information.

5. The application of claim 1, wherein the second user portal is configured to share data with non-users.

6. The application of claim 1, wherein the second user portal is configured to share automatically generated insight with the first user.

7. The application of claim 1, wherein the second user portal further comprises one or more task buttons wherein each task button automatically triggers one or more tasks when selected.

8. The application of claim 1, wherein the report is editable.

9. The application of claim 1, wherein the report comprises text and audiovisual content.

10. The application of claim 1, wherein the report comprises text and audio content.

11. The application of claim 3, wherein the symptom information is input by a user through selection from a preset symptoms list.

12. A method of improving the quality and speed of physician review of data, the method comprising:
a) receiving data for viewing on a single review screen;
a) associating one or more non-opaque classification buttons to the data wherein said association of the one or more opaque classification buttons automatically generates a report; and
b) selecting one or more non-opaque task buttons wherein each task button automatically triggers one or more tasks when selected.

13. The method of claim 12, further comprising the steps of editing the diagnosis report and sharing the diagnosis report with a first user.

14. The method of claim 13, wherein the data comprises bioelectrical data.

15. The method of claim 13, wherein the data comprises bioelectrical data and symptom information.

16. The method of claim 13, wherein the symptom information is input by a user through selection from a preset symptoms list.

17. The method of claim 13, wherein the report comprises text and audiovisual content.

18. The method of claim 13, wherein the report comprises text and audio content.

19. A method of improving the quality and speed of physician review of data, the method comprising:
b) receiving a list of multiple patients’ data for review in order of priority;
c) accessing an individual patient’s data for viewing on a single review screen;
c) associating one or more non-opaque classification buttons to the data wherein said association of the one or more opaque classification buttons automatically generates a report; and
d) selecting one or more non-opaque task buttons wherein each task button automatically triggers one or more tasks when selected.
